(19) United States
(12) Patent Application Publication
(10) Pub. No.: US 2026/0325344 A1
(43) Pub. Date: Oct. 22, 2026

(54) ADJUSTABLE DEVICE FOR ORGAN PROLAPSE

(71) Applicant: Pessarease Ltd., Netanya (IL)

(72) Inventors: Amnon WEICHSELBAUM, Haifa (IL); Itamar DACH, Pardes Hanna (IL); Roy Zion MANSANO, Encino, CA (US)

(21) Appl. No.: 19/724,124

(22) Filed: Jun. 29, 2026

Related U.S. Application Data
(63) Continuation-in-part of application No. PCT/IB2024/063260, filed on Dec. 29, 2024.
(60) Provisional application No. 63/615,799, filed on Dec. 29, 2023.

(51) Int. Cl. A61F 6/08; A61F 2/00

(52) U.S. Cl. CPC A61F 6/08; A61F 2/005

(57) ABSTRACT

A vaginal apparatus includes a cervical cap configured for mounting in a cervix, and a vaginal support unit configured for installation in a vaginal canal and which has an upper end extending from the cervical cap. The vaginal support unit has expandable and collapsible arcuate support members formed with perforations. In some embodiments of the apparatus, said arcuate support members each have a composite structure comprising a flexible polymeric body and an internal support framework formed from a shape-memory material.

### FIELD OF THE INVENTION

The present invention relates to a self-inserted, vaginal apparatus, such as for treating pelvic organ prolapse (POP).

### BACKGROUND OF THE INVENTION

The pelvic floor is comprised of a group of muscles, ligaments, and connective tissues that collaborate to provide support for the pelvic organs. In females, these muscles create a supportive sheet, maintaining the bladder, rectum, and uterus in their respective proper positions. These muscles are affixed to the stable framework of the pelvic bones. Pelvic organ prolapse (POP) occurs when the pelvic floor muscles weaken, allowing one or more of these pelvic organs to exert pressure against the weakened vaginal wall. While POP is typically not life-threatening, it can impact urinary, sexual, and colorectal functions. For women experiencing significant symptoms, the current management of POP involves either surgical reconstruction or the use of vaginal pessaries.

A pessary for prolapse is a medical device designed to provide support and alleviate symptoms associated with pelvic organ prolapse. The pessary is inserted into the vagina to help support and reposition displaced organs, such as the uterus. Pessaries come in various shapes and sizes, and their selection depends on the specific type and severity of the prolapse. They work by providing mechanical support to the pelvic organs and tissues, thereby improving symptoms such as discomfort, pressure, and urinary issues caused by pelvic organ prolapse. Pessaries are typically recommended after a thorough evaluation by a healthcare professional to ensure proper fitting and effectiveness.

A variety of pessaries are currently available for the management of pelvic organ prolapse, each designed to address different types and degrees of prolapse. Some of the most common types include Ring Pessary, Gellhorn Pessary, Cube Pessary and Inflatable Pessary.

The healthcare provider will select the pessary based on patient anatomy, type and severity of prolapse, comfort and efficacy. It’s essential for a healthcare professional to properly fit and monitor the use of a pessary to ensure its effectiveness and the patient’s well-being.

### SUMMARY OF THE INVENTION

The present invention seeks to provide a self-inserted, adjustable vaginal apparatus, such as a universal support for treating pelvic organ prolapse (POP), as is explained more in detail below. Although the invention is described with reference to a vaginal supporting apparatus, the invention can be used to support other structures, such as the urinary bladder and rectum.

A collapsible vaginal pessary designed for the management of pelvic organ prolapse is disclosed. The apparatus includes means for orientation, attachment and a mechanical support to the cervical and vaginal tissue for supporting thereof by applying counter pressure to the vaginal walls in order to prevent pelvic organ prolapse.

In one aspect, the present invention is directed to a vaginal apparatus comprising: a cervical cap configured for mounting in a cervix; and a vaginal support unit configured for installation in a vaginal canal and which has an upper end extending from said cervical cap, said vaginal support unit comprising expandable and collapsible arcuate support members. In some embodiments, said arcuate support members comprise perforations.

In certain embodiments, the apparatus comprises a composite structure wherein the arcuate support members comprise a flexible polymeric body and an internal support framework formed from a shape-memory material, as will be discussed in more detail, hereinbelow.

In some embodiments of the apparatus, the annular rim frame is coupled to a lower portion of the arcuate support members. In some cases, an outside surface of said annular rim frame is lined or covered by a friction enhancing material.

In some preferred embodiments, the arcuate support members are shaped as petals or wings. In some cases, these support members have rounded ends and edges. In still other embodiments said arcuate support members have inward-oriented ends. In other cases, the arcuate support members are coupled to a central shaft.

The aforementioned cervical cap, in some embodiments, comprises circumferential cervical interface members, each of which has radial sides made of an upper straight portion from which extends an arcuate portion, and wherein said cervical interface members are separated at upper ends of the upper straight portions by circumferential gaps.

### BRIEF DESCRIPTION OF THE DRAWINGS

The present invention will be understood and appreciated more fully from the following detailed description, taken in conjunction with the drawings in which:

FIG. 1 is a simplified perspective illustration of the vaginal apparatus inserted in a vagina, in accordance with a non-limiting embodiment of the present invention.

FIG. 2 is a simplified perspective illustration of the structure of the vaginal apparatus, in accordance with a non-limiting embodiment of the present invention.

FIG. 3 is a simplified perspective illustration of an alternative structure of the vaginal apparatus, in accordance with another non-limiting embodiment of the present invention.

FIGS. 4A, 4B, 4C and 4D are simplified front perspective, distal end view, rear perspective, and proximal end view illustrations, respectively, of a vaginal apparatus, in accordance with another non-limiting embodiment of the present invention.

FIG. 5A and 5B provide, respectively, a top view and side view of one preferred embodiment of the vaginal apparatus of the present invention, in which the support unit is constructed from a flexible polymer body and an internal support framework formed from a shape-memory material.

### DETAILED DESCRIPTION

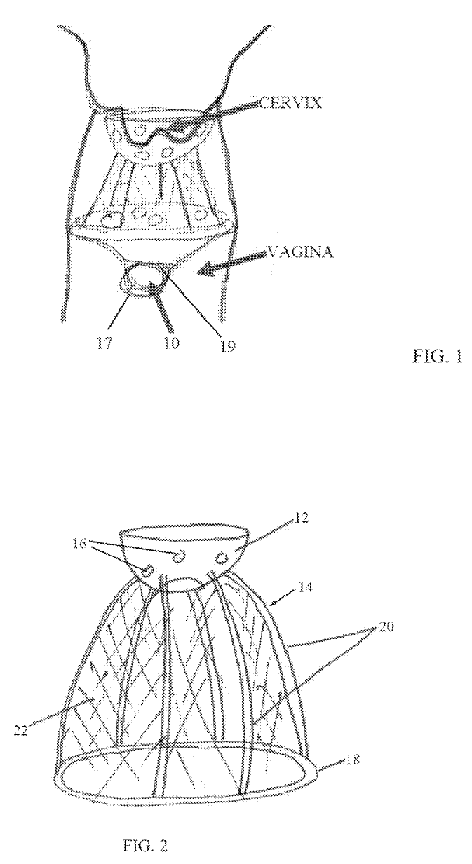

Reference is now made to FIGS. 1-3, which illustrate a vaginal apparatus 10, in accordance with a non-limiting embodiment of the present invention.

The vaginal apparatus 10 may include a cervical cap 12 (the upper unit when installed) and a vaginal support unit 14 (the lower unit when installed; also called a skirt), configured for installation in a vaginal canal. The cervical cap 12 may include a pliable and resilient cap tailored to the dimensions of the cervix and encompassing it. The cervical cap 12 may include one or more perforations 16. The vaginal support unit 14 may be constructed as a skirt that effortlessly expands upon insertion into the patient’s vagina, securely anchoring itself within the vaginal cavity during movement to effectively counteract the effects of organ prolapse and increased intraabdominal pressure.

Without limitation, the vaginal support unit 14 may include a rim frame 18, which is affixed to the bottom of arcuate support members 20, referred to as arcuate ribs 20. The rim frame 18 is elastic and is foldable and compressible, facilitating the insertion of the device into the vagina. Once in position, the frame unfolds and outwardly exerts radial pressure on the vaginal walls, securely anchoring the device.

The arcuate ribs 20, which may be semi-rigid, couple the rim frame 18 to the cervical cap 12. The ribs 20 may be constructed as arc-shaped vertical struts (AVS). The AVS structure of the ribs offers extra spring-like resilience to aid in the semi-automatic deployment of the device into an expanded position within the vaginal cavity.

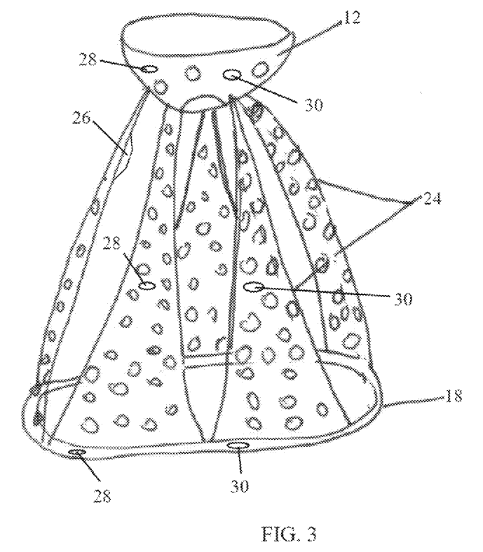

The ribs 20 can be in the form of elongated rods with a perforated membrane 22 connecting them, or alternatively a triangular perforated structure 24 (FIG. 3) whose base is at the caudal end of the vagina. The outside surface of the elastic rim may be lined or covered by a friction enhancing material, such as a ribbed pattern silicone material to prevent slippage of the device from the vagina.

The combination of the cervical cap 12 and the vaginal support unit 14 allows for both vertical and horizontal pressure to be exerted on the vagina by the pelvic organs. Downward pressure from the cervix is supported by the ribs 20, which remain intact and which transfer this pressure to the ribs 20 of the skirt 14. This pressure causes the ribs 20 to expand outwards and press against the vaginal walls, thereby securing the device in the vagina and preventing uterine descent. Similarly, lateral pressure from the bladder and/or rectum will be supported by the counter pressure of the ribs 20 on the contralateral vaginal wall, thus providing lateral support.

Accordingly the vaginal apparatus 10 supports and maintains the vaginal structural integrity, with the vaginal support unit 14 applying supportive pressure on the vaginal walls, thus gently lifting and supporting the urethra. Thus, the device may also treat stress urinary incontinence.

A retrieval string 17 or silicone ring 19 (FIG. 1) coupled to the rim frame of the skirt, may be pulled to collapse the device for easy removal of the device from the vagina.

To treat the vaginal tissue, the device can incorporate one or more compartments 26 (FIG. 3) containing fluid or gel materials, such as hormones, which are slowly and gradually released into the vaginal cavity.

One or more sensors 28 or stimulators 30 (FIG. 3) may be provided on a portion of the apparatus 10 for electrical stimulation or sensing, such as monitoring the collapse of vaginal walls (including the uterine fornix), pH sensing, or other sensor functionalities.

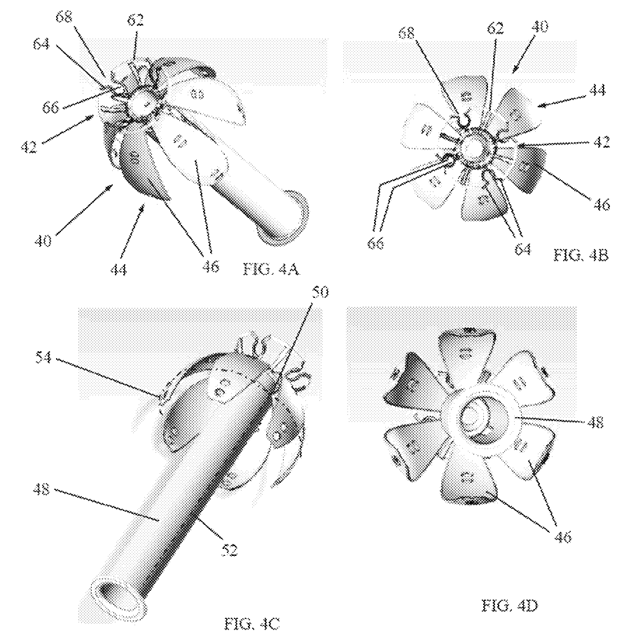

Reference is now made to FIGS. 4A-4D, which illustrate a vaginal apparatus 40, in accordance with another non-limiting embodiment of the present invention, including a winged skirt design for enhanced functionality and user convenience.

The vaginal apparatus 40 may include a cervical cap 42 (the upper unit when installed) and a vaginal support unit 44 (the lower unit when installed; also called a skirt). The cervical cap 42 may include circumferential cervical interface members 62, each of which has radial sides made of an upper straight portion 64 from which extends an arcuate portion 66. The cervical interface members 62 are separated at the upper ends of the upper straight portions 64 by circumferential gaps 68. Adjacent upper straight portions 64 slant away from each other to form a Y-shape and adjacent arcuate portions 66 form a U-shape.

Without limitation, the vaginal support unit 44 may include arcuate support members 46, shaped as flexible petals or wings. The arcuate support members 46 can be made from silicone, polypropylene, or any other medical-grade material designed to meet the requirements of being biocompatible and flexible, while still providing adequate radial pressure to support various types of prolapses. These materials are carefully selected to avoid causing tissue damage or irritation and to ensure the device remains securely in place, preventing expulsion.

The vaginal apparatus of all the embodiments of the invention may comprise components made from various biocompatible materials, including but not limited to silicone, polypropylene, thermoplastic elastomers, hydrogels, or other medical-grade materials suitable for flexibility, durability, and patient safety. As explained hereinabove, in some embodiments the apparatus may be constructed from a flexible polymer body and an internal support framework formed from a shape-memory material. More details concerning the structure of these embodiments, and suitable materials for their construction are provided hereinbelow.

The arcuate support members 46 may have petals or wings with rounded ends and edges, which create an effective friction and support area, while at the same time preventing tissue damage and minimizing the risk of the device slipping out.

Alternatively, the arcuate support members 46 may have inward-oriented ends. This orientation further reduces the risk of tissue damage and enhances device stability.

Each arcuate support member 46 may be coupled to a central shaft 48, such as by means of struts, bands or strings 50 (FIG. 4C), to create an umbrella or folding mechanism, which can be actuated by a pushing or pulling member 52 (shown partially in broken lines in FIG. 4C). Movement of the pushing or pulling member 52 either deploys the arcuate support members 46 radially outwards from the central shaft 48 or collapses the arcuate support members 46 radially inwards towards the central shaft 48.

Alternatively, the arcuate support members 46 may be resilient or spring-biased members, which are initially held by a ring 54 (shown partially in broken lines in FIG. 4C) in the collapsed position, and upon removal of the ring, expand outwards to the deployed position. In order to remove the device, a retrieval ring may be connected to the central shaft and the struts, bands or strings. The retrieval ring can be pulled to reapply tension to the struts, bands or strings, thereby collapsing the arcuate support members 46 radially inwards towards the central shaft 48.

The folding mechanism may be based on alternate systems, including but not limited to, pneumatic, hydraulic, or magnetically-assisted mechanisms, to improve deployment and usability.

The folding mechanism may be an applicator that expands the arcuate support members 46 after inserting the device into the vagina. The applicator may then be detached from the apparatus 40 to release the apparatus which remains in the vagina. The applicator may be reattached to the device to collapse the arcuate support members 46 for withdrawing the apparatus from the vagina.

Once the device is correctly positioned, indicated by the cervical cap 42 encircling the cervix, the applicator may release the device in place and be withdrawn.

The vaginal apparatus and associated components may include anti-slip coatings, antibacterial material properties, and fail-safe mechanisms to prevent accidental deployment or removal.

The vaginal apparatus may optionally include embedded sensors to monitor positioning, pressure applied to surrounding tissues, or other relevant parameters for realtime feedback to medical professionals or users.

In certain embodiments, the vaginal support unit comprises a composite structure including a flexible polymeric body and an internal support framework formed from a shape-memory material. The flexible polymeric body may be formed from silicone, thermoplastic elastomers (TPE), polyurethane, hydrogel-based materials, or other biocompatible medical-grade polymers. The internal framework may comprise Nitinol (nickel-titanium alloy) or other shape-memory alloys, shape-memory polymers, superelastic materials, resilient metallic structures, or combinations thereof.

In a preferred embodiment, a Nitinol framework is embedded within, overmolded into, or threaded through the silicone support members or petals of the device. The shape-memory framework provides structural reinforcement while preserving the flexibility and atraumatic characteristics of the surrounding polymeric material.

The internal framework may be configured to provide anisotropic mechanical behavior whereby resistance to deformation differs according to the direction of applied load. Such anisotropic support may preferentially resist prolapse-associated load vectors while preserving flexibility during insertion, removal, ambulation, voiding, defecation, and other normal physiologic activities.

This dual-material architecture provides combined rigidity and flexibility, creating a dynamic support structure capable of responding to changing anatomical loads. Unlike purely flexible support devices, the shape-memory framework actively resists deformation and generates adaptive counterforces in response to pressure exerted by prolapsing pelvic organs. Such pressures may originate from the uterus, bladder, rectum, vaginal walls, or any combination thereof.

Upon deformation by prolapsing tissue, the framework may accumulate elastic strain energy and generate a restoring force tending to return the device toward a predetermined deployed configuration.

In certain embodiments, the support framework exhibits progressive or nonlinear stiffness such that resistance to deformation increases as displacement increases, thereby enhancing patient comfort during routine activity while providing increased support under higher prolapse loads.

In certain embodiments, deformation of the support structure by prolapsing pelvic organs causes the shape-memory framework to transition from a relatively compliant state to a relatively supportive state, thereby providing load-dependent support. The magnitude of the restoring force may increase as prolapse severity or displacement increases, creating a self-adjusting support mechanism that accommodates normal physiologic motion while resisting progressive organ descent.

The shape-memory framework enables the device to dynamically alter its support characteristics according to the magnitude and direction of the applied load vectors, thereby improving support for different forms and severities of pelvic organ prolapse. In this way, the range of application of the device of the present invention to these various types of prolapse can be significantly increased, when compared with prior art devices.

The framework may dynamically respond to transient increases in intra-abdominal pressure associated with coughing, sneezing, lifting, exercise, Valsalva maneuvers, defecation, or other physiologic activities. Simultaneously, the surrounding silicone or other soft polymeric material distributes contact forces over a larger surface area, reducing localized pressure concentrations and minimizing the risk of tissue irritation, erosion, ulceration, or discomfort.

Accordingly, the combination of a flexible polymeric body and an internal shape-memory support framework provides enhanced anatomical conformity, improved retention, adaptive support under varying physiological conditions, and increased patient comfort compared with structures formed solely from flexible materials.

Following compression for insertion, the framework may automatically recover toward its predetermined deployed configuration upon release, thereby facilitating atraumatic insertion and reliable deployment.

The framework may maintain substantially unchanged support characteristics following repeated cyclic loading over prolonged implantation periods, thereby improving durability and long-term performance.

The framework may be manufactured by laser cutting, wire forming, additive manufacturing, or other suitable techniques, and may subsequently be assembled with the polymeric body by insert molding, overmolding, encapsulation, mechanical engagement, adhesive bonding, or combinations thereof.

The framework may comprise one or more circumferential rings, radial members, lattice structures, mesh structures, braided structures, helical structures, serpentine elements, petal reinforcements, or combinations thereof.

In some embodiments, the device may have an overall deployed diameter of approximately 30–100 mm, a collapsed insertion diameter of approximately 10–35 mm, and a longitudinal length of approximately 20–120 mm, although other dimensions may be utilized depending on the patient's anatomy and the intended clinical application.

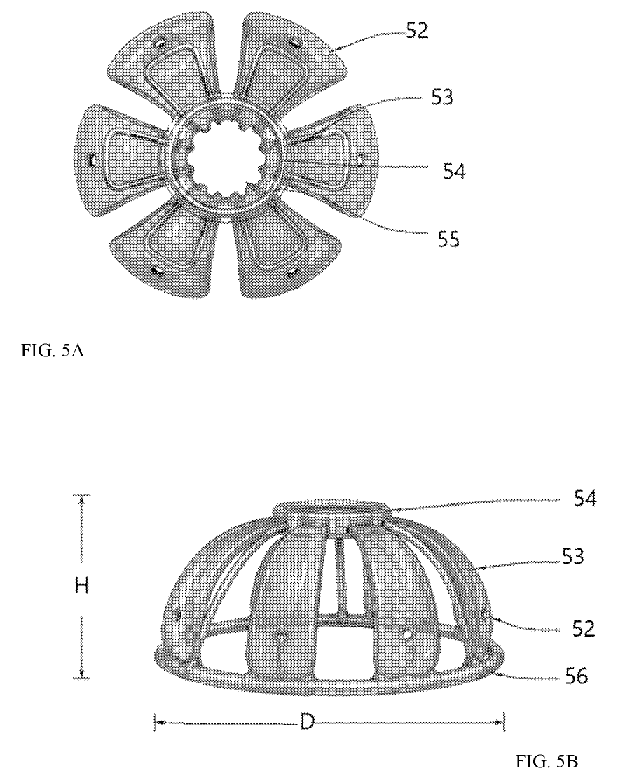

One example of an embodiment of the device of the present invention having the composition silicone-Nitinol construction is shown in its open, deployed configuration in FIGS. 5A (top view) and 5B (side view). The device, as illustrated comprises a Nitinol framework 53 embedded within silicone petals (or wings) 52, which pass inferiorly from cervical cap 54 (constructed of silicone) to rim frame 56 (also silicone). The device is also constructed such that openings for tissue adaptation 55 are formed between adjacent petals. As shown in the side view in FIG. 5B, this embodiment, in its deployed configuration, has a height H, which is typically in the range of about 30 – 60 mm, and an external diameter D, typically in the range of about 40 – 90 mm.

1. A vaginal apparatus comprising:
a cervical cap configured for mounting in a cervix; and
a vaginal support unit configured for installation in a vaginal canal and which has an upper end extending from said cervical cap, said vaginal support unit comprising expandable and collapsible arcuate support members formed with perforations.

2. The vaginal apparatus according to claim 1, wherein the arcuate support members each comprise a flexible polymer body and an internal support framework formed from a shape-memory material.

3. The vaginal apparatus according to claim 1, wherein an annular rim frame is coupled to a lower portion of said arcuate support members.

4. The vaginal apparatus according to claim 3, wherein an outside surface of the annular rim frame is lined or covered by a friction enhancing material.

5. The vaginal apparatus according to claim 1, wherein said expandable and collapsible arcuate support members are shaped as petals or wings.

6. The vaginal apparatus according to claim 1, wherein said cervical cap comprises circumferential cervical interface members, each of which has radial sides made of an upper straight portion from which extends an arcuate portion, and wherein said cervical interface members are separated at upper ends of the upper straight portions by circumferential gaps.

7. The vaginal apparatus according to claim 5, wherein said arcuate support members have rounded ends and edges.

8. The vaginal apparatus according to claim 5, wherein said arcuate support members have inward-oriented ends.

9. The vaginal apparatus according to claim 1, wherein said arcuate support members are coupled to a central shaft.
